(19) United States
(12) Patent Application Publication
(10) Pub. No.: US 2024/0024065 A1
(43) Pub. Date: Jan. 25, 2024

(54) Method and Platform for Portable Remote Support During Medical Device Implantation Using Augmented Reality Devices

(71) Applicant: Orcana Inc., Carmel-by-the-Sea, CA (US)

(72) Inventors: Kris Okumu, Millbrae, CA (US); Orr Orenstein, San Francisco, CA (US)

(73) Assignee: Orcana Inc., Carmel-by-the-Sea, CA (US)

(21) Appl. No.: 18/041,403

(22) PCT Filed: Aug. 10, 2021
(86) PCT No.: PCT/US2021/045454

§ 371 Date: Feb. 10, 2023

Related U.S. Application Data
(60) Provisional application No. 63/063,868, filed on Aug. 10, 2020.

(51) Int. Cl. A61B 90/00; G06T 11/00; G06F 3/01; G06V 20/20; G06V 20/50; G06T 13/00; H04L 65/403; G16H 80/00; G16H 20/40

(52) U.S. Cl. CPC A61B 90/37; G06T 11/00; G06F 3/017; G06V 20/20; G06V 20/50; G06T 13/00; H04L 65/403; G16H 80/00; G16H 20/40; G06V 2201/03; G06T 2210/41; A61B 2090/365

(57) ABSTRACT

A framework for medical and surgical support enables remote guidance for implantations of medical devices during medical procedures and integrates video conferencing technology to enable product support specialists to provide support to medical personnel for such implantations in an operating facility during interactive collaboration sessions. The framework enables individuals wearing augmented reality-enabled display devices to communicate in real time with such product support specialists for medical devices who are not physically present in an operative theater, to promote and maintain sterile environments and protect the health and safety of the various participants in such collaboration sessions. The framework includes real-time, secure communication capabilities from, to, and between such medical personnel and any product support specialist client. The framework further includes hardware components and software elements that enable gesture detection and interpretation. The framework also enables instruments and devices to be tracked, monitored, and verified before, during, and after medical procedures using image processing techniques that allow for object detection and recognition.

This application is a 35 U.S.C. § 371 U.S. national stage patent application which claims the benefit of priority and is entitled to the filing date of International Patent Application PCT/US2021/045454, filed Aug. 10, 2021, an international patent application which claims the benefit of priority and is entitled to the filing date pursuant of 35 U.S.C. § 119(e) of U.S. Provisional Patent Application 63/063,868, filed Aug. 10, 2020, the content of each of which is hereby incorporated by reference in its entirety.

### FIELD

The present invention relates to the field of medical support and training systems. Specifically, the present invention relates to a method for remotely guiding implantation of a medical device during a surgical procedure, and for remote guidance during medical procedures generally, and a collaborative tool that enables remote conferencing in conjunction with wearable augmented reality-configured displays for supporting activities in medical environments. Additional applications and supported use cases include remote physicians overseeing, guiding, and supervising on-site surgeons and associated personnel, training of surgical physicians and associated personnel on activities such as the implantation of devices through remote training guidance, and inventory management for hospitals and other facilities where implantations of medical devices and other surgeries and procedures are performed.

### BACKGROUND

Current medical device technology has greatly enhanced the healthcare field. Such technology assisting medical professionals during both invasive and non-invasive surgeries and other medical procedures have contributed to improved patient outcomes as well as the overall level of medical care provided. For example, sophisticated medical devices, such as, pacemakers, cochlear implants, prosthetics, hip replacements, and other medical implants, function to impart or enhance function of a damaged organ or tissue, replace a missing body part, deliver medication, monitor body functions, or provide support to organs and tissues.

Other devices provide image-guided and/or computer-navigated surgical support allowing surgeons and other medical professionals to perform many surgical procedures safely by avoiding invasive and risky medical procedures. For example, cameras may be affixed to surgical instruments, allowing medical professionals to view images collected by those instruments during operation within a patient's body. Additionally, such instruments can include operational components that enable the operator to remotely obtain tissue samples or perform surgical repair non-invasively.

Due to the complex nature of current medical devices and the associated procedures required to properly implant these devices, a product support specialist, also known as a medical device representative, for a particular medical device is typically present during the surgical procedure. A product support specialist is trained by the company manufacturing the medical device and most have observed significantly more procedures using the medical device than the surgeon performing the surgery. Such a specialist aids the surgeon by providing technical assistance in the assembly and/or performance of a medical device as well as best practices and procedures regarding the implantation or use of such a device. In addition, given the knowledge and familiarity of the medical device, a product support specialist assists in monitoring the components of a medical device and inventorying such components to ensure none are lost or inadvertently left within the patient after the surgery is completed.

Currently, such product support specialists must be in the same surgical suite as the physician, whether in the actual operating theater standing near the surgeon, or in an adjacent room allowing visual observation of the medical procedure as well as audio communication with the surgeon. While the presence of a product support specialist is advantageous to the surgeon and operating staff, there are disadvantages as well. For example, communication between the surgeon and a product support specialist regarding a complex subject or procedure may be needed during the procedure. Such communication often leads to the surgeon having to shift attention away from the patient to focus on the product support specialist or a monitor displaying critical information.

Additionally, the presence of a product support specialist in the operating room increases liability to the health of a patient due to an increased risk of contamination, such as infection due to a pathogen. Due to the current ongoing COVID-19 pandemic, such concerns have heightened, and hospitals have imposed restrictions on the movement of individuals in operating rooms, and within hospitals in general. Neither patients nor anyone working during a procedure in close proximity to others want to become inadvertently infected by an individual involved who does not know they are carriers of dangerous viruses or bacteria for which there are no current treatments available.

Thus, a conundrum currently exists in that while it has become riskier to have medically unnecessary people present inside a hospital and inside an operating theater, at the same time, the complexity of medical instruments and equipment involved in making the actual procedures safer and more effective may require support from individuals such as product support specialists who are not directly involved in a procedure.

Therefore, there is a need in the existing art for an approach that enables remote, real-time participation and collaboration during implantation and/or use of a medical device between a surgeon, and those working near them, and a product support specialist or other individuals external to the operating theater. Such an approach should maintain, if not enhance, the current benefits provided by a product support specialist while reducing, if not eliminating, the disadvantages currently associated with the specialist's presence in the operation room. In addition, the approach should improve overall safety for both the patient and for those involved in a medical procedure, reduce risks due to issues such as contamination and incomplete or damaged instruments, and minimize distractions from those performing a procedure due to having to look away and focus attention on a monitor or device. Furthermore, there is a need for an approach that independently tracks, monitors, and verifies instruments and materials used in a medical or surgical procedure both before and after such procedures.

### SUMMARY

The present invention addresses these issues in an architecture for remote medical and surgical support for implantation of medical devices, and associated hardware and software framework, and systems, methods and tools that embody and implement such an architecture and associated framework and integrate specific hardware elements such as augmented reality devices and head-up displays, as well as software elements that enable internal and external personnel to collaborate via telepresence using such hardware elements. The present invention includes a plurality of systems, methods, and tools for remote, real-time attendance during a medical procedure of a product support specialist, or other individuals, external to the operating theater and enable remote, real-time, multimedia communication and other information sharing capabilities between such a specialist and a surgeon, or other in-theater personnel, who are performing, or assisting in, the medical procedure using such augmented reality devices. These systems, methods, and tools operate in conjunction with wearable head-up displays such as those enabled with augmented reality capabilities, permitting an entire team of people to observe from the surgeon's perspective, what a surgeon is viewing and provide real-time support. Such capabilities allow a surgeon, or person using the hardware, and those working near them to avoid having to look away from the patient on whom a procedure is being performed.

The architecture and framework provide systems, methods, and tools for network-based collaboration and integration of content that is displayed and consumed in many different forms, such as (but not limited to) text, annotations, audio, images, animations, and video, as well as interactive involvement between participants and such content. The architecture and framework provide an interactive platform over which multiple individuals can participate in a collaboration session during the performance of procedures, view and manipulate information in various content forms as noted above and communicate with each other in real-time. The architecture and framework also include and operates with multiple hardware devices that at least include displays over which information can be captured and viewed during a collaborative session, such as wearable augmented reality headsets or other additional cameras/screens at the procedure site.

The architecture and framework also enable sterile, hands-free, voice and gesture-controlled, remote-support multimedia conferencing using, as noted above, augmented reality devices and other devices that enable displays of images, graphics, and videos, as well as communications between participants during sessions. In one aspect thereof, the architecture seamlessly enables a surgeon, “scrub” tech, nurse, or other operating assistant in an operating room to receive support and share full multimedia communication to and from remotely situated product support specialists. The architecture and framework include client-server text, audio, video streaming and sharing software, and security through private wireless network connectivity, Wi-Fi, and 4G/5G cellular networks between display devices, the conferencing server(s), and any additional attendee clients or administrative control panels.

The architecture and framework further include situational awareness capabilities that allow remote medical device representatives to understand commands from personnel inside an operating room communicated by gestures. The architecture and framework therefore interpret gestures made by wearers of augmented reality devices and enables gesture control of actions within a session to be understood by such remote product representatives.

The architecture and framework also provide for remotely ensuring that an implant is working correctly, acquiring data from an implant remotely, and remotely programming an implant, such as for example a pacemaker.

The architecture and framework also provide an independent approach for tracking, monitoring, and verifying components of the medical device and instruments used in a medical or surgical procedure, both before and after those procedures are performed. For example, such inventory control capabilities of the present invention disclosed herein can ensure that all components of a medical device as well as all surgical tools and other medical instruments needed for the procedure are present, sterilized, and ready for use before the procedure is initiated. Likewise, a similar inventory control process is performed in real-time during the procedure as well as after the procedure is completed and not mistakenly left inside a patient's body. In this aspect of the present invention, the architecture and framework also include image detection capabilities or other techniques for pixel characterization that enable an automated detection and identification of such components and instruments in an operating facility, as well as providing alerts or notifications that such instruments are present and ready for use, or in the alternative, missing and need to be provided or reordered for future procedures. Such inventory control capabilities provide an independent check that does not rely on individuals manually counting and verifying that all items are present and accounted for.

It is therefore one objective of the present invention to provide a system, method, and tools for remote collaboration in a medical or surgical setting. It is another objective of the present invention to provide a system, method and tools for such remote collaboration that includes the ability to view and interact with content in different forms and enable communications between collaborators in the medical or surgical setting during a session. It is still another objective of the present invention to provide a system, method, and tools for participation of product support specialists in a medical or surgical procedure involving implantation of medical devices, where the product support specialists provide remote guidance for such an implantation to the operating team involved in such a procedure. Additional objectives include scenarios for the remote support of on-site physicians, surgeons, and other associated personnel to be supervised or guided by offsite remote observers, such as superiors and attending physicians, and finally, the same system supports the objective of enabling the remote training of physicians, surgeons, and associated personnel, who would otherwise need to travel for a physical training, to be certified and trained remotely through the use of this invention.

It is a further objective of the present invention to provide a system, method, and tools of wirelessly enabling such remote collaboration, content consumption, and communication. It is still further an objective of the present invention to provide a system, method, and tools in which such remote session collaboration is integrated with devices such as wearable augmented reality headsets and other devices that enable display of images, video and other multimedia forms of communication.

It is another objective of the present invention to address and minimize risks associated with invasive medical and surgical procedures using equipment that either requires support or involves the use of additional devices, or both. It is another objective of the present invention to address such risks by reducing the number of people that need to be present in an operating facility, to reduce traffic in and out of, and within, the operating facility. It is still another objective of the present invention to address such risks by reducing the need for additional, bulky equipment in the operating facility. It is still another objective of the present invention to address such risks specifically as to product support specialists by reducing exposure to radiation to such product support specialists and reducing a risk of viral transmission to/from such product support specialists, by eliminating the need for their physical presence in the operating facility itself. It is yet another objective of the present invention to provide a support framework for personnel working in an operating facility that promotes and maintains a sterile, hygienic, and clean environment. It is still another objective of the present invention to provide an approach for independently tracking, monitoring, and verifying materials, instruments, and devices involved in procedures such as implantation of medical devices, to ensure that all materials are present and prepared before procedures and accounted for after procedures.

Still further objectives of the present invention include always ensuring availability of product and surgical support at all times during a procedure, reducing a patient's risk of surgical site infection, reducing travel time and expenses of product support specialists, and improving patient privacy.

Other objects, embodiments, features, and advantages of the present invention will become apparent from the following description of the embodiments, taken together with the figures below, which illustrate, by way of example, the principles of the invention.

### BREIF DESCRIPTION OF THE DRAWINGS

FIG. 1 is a system diagram illustrating exemplary hardware elements and connectivity between such elements within architecture for remote medical and surgical support, and an associated hardware and software framework for remote guidance of medical device implantation during medical or surgical procedures disclosed herein;

FIG. 2 is a further diagram illustrating an exemplary high-level architecture comprised of personnel at a first location and relative to an operating theater and at a remote, second location; and

FIG. 3 is a further diagram illustrating exemplary local hardware components of the architecture and framework for remote guidance of medical device implantation during medical or surgical procedures at a first location.

### DETAILED DESCRIPTION

In the following description of the present invention, reference is made to the exemplary embodiments illustrating the principles of the present invention and how it is practiced. Other embodiments will be utilized to practice the present invention and structural and functional changes will be made thereto without departing from the scope of the present invention.

The present invention is an architecture for remote medical and surgical support, and an associated hardware and software framework 100 (referred to herein simply as a “framework”), for remote guidance of medical device implantation during medical or surgical procedures, that integrates video calling and conferencing technology with software that enables on-site or remotely-stationed product support specialists to view, interact with, communicate with, and provide support to medical personnel in an operating facility or theater, at least one of whom is using or wearing an augmented reality-enabled display (also referred to herein as a “head-up display” or “HUD”). The framework 100 includes all components necessary for real-time communication (including audio, video, text and screensharing, and annotations for screenshots and live feeds) from, to and between such an augmented reality-enabled display and any product support specialist client connected to one or more local, remote servers and used by product support specialists (also referred to herein as “PSSs”), and on a particular secure channel for such communication. The framework 100 also includes further components which enable features that are required specifically for the product support specialists, and still others required for operation of the HUD, such as gesture detection and control activation. The framework 100 is built on an extensible, portable platform that allows for additional features, modules, and components to be added at a later time and/or in future embodiments of the present invention.

The solution of the framework 100 provides client-server text, audio, and video streaming and sharing software technology, a heads-up display (HUD), one or more tablet and mobile device with security through private wireless network connectivity between the HUD, the conferencing server, and any additional attendee clients or administrative control panels. Each of these elements of the present invention enables product support specialists to remotely guide medical professionals during medical or surgical procedures involving implantation of medical devices having vantage points from one of several points of view.

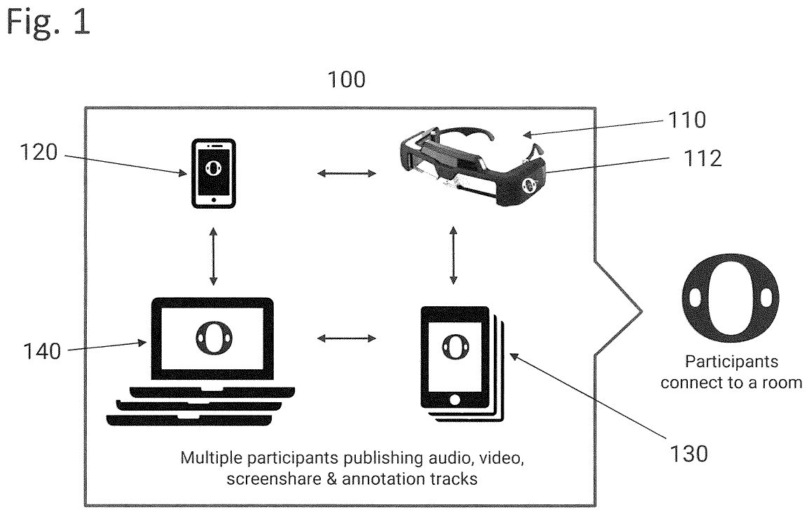

FIG. 1 is a systemic diagram showing the general connectivity among devices within the framework 100 for remote guidance of medical device implantation during medical or surgical procedures of the present invention. The framework 100 of the present invention includes networking features to ensure secure and reliable operation thereof. Due to limited network connectivity available in hospitals and operating rooms, and/or to inaccessible local Wi-Fi networks due at least in part to the locations of such facilities or the presence of other equipment, the framework 100 is available in different networking configurations, and it is to be understood that the present invention may therefore operate in different networking environments and may comprise different configurations. Two such configurations are listed below, but it is to be further understood that this disclosure is not to be limited to any one type of configuration mentioned here, and that other configurations are possible and within the scope of the present invention. Irrespective of the networking configuration within which the present invention is operable however, it is to be understood that the framework 100 does not require an existing network in a hospital or ambulatory surgery center where a medical or surgical procedure is to be performed and operates entirely as a closed system to reduce system vulnerability and improve security and reliability.

In one such networking configuration, the framework 100 operates within a secure, private network, supported via a fully local, on-site server, using Wi-Fi for connectivity between components. In this configuration, a fully self-contained local server is utilized, with an access point included for secure point-to-point connections. Any number of augmented reality HUD clients, PSS clients, tablets, mobiles, or other clients may connect to this secure private network. Product support specialists may be on-site, yet still away from, or outside of, the actual operating facility, as long as they are within Wi-Fi signal range to connect to clients in the actual operating facility. Specific HUD connectivity features are available exclusively in this configuration, including default network identification and access and device auto-registration to a local server 160. Such a secure, private network is not dependent on hospital or facility communications infrastructure and operates as a closed network separate from any hospital servers or hospital Wi-Fi connectivity.

In another configuration, the framework 100 operates within a cloud-hosted network infrastructure, so to be supported via server components that are hosted in a cloud computing environment. This configuration is operable where adequate (or trusted) network connectivity is available, and support is being provided by a remote, physically off-site product support specialist, or an on-site product support specialist without access to a local server. This may be facilitated though the on-site Wi-Fi network or cellular 4G/5G connectivity.

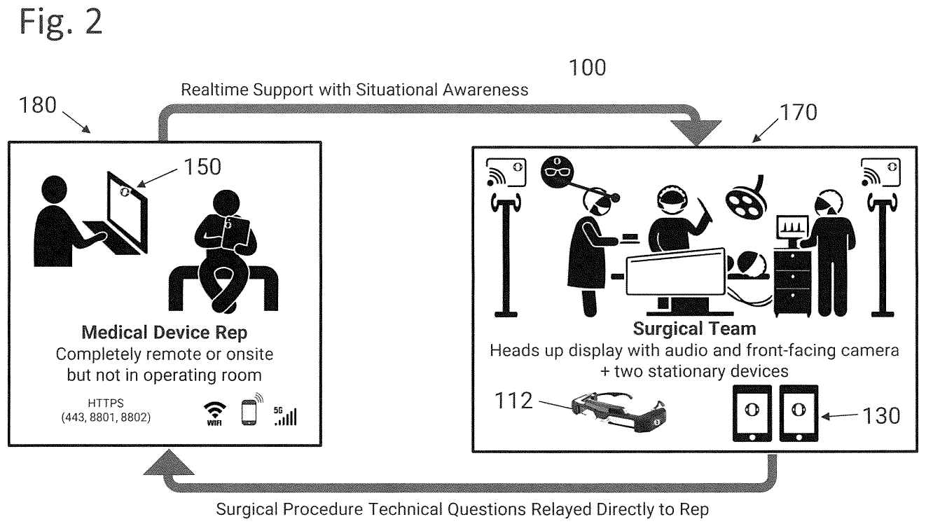

Returning to FIG. 1, as noted above, the present invention is a remote support system and accompanying software within the framework 100 for use in, according to one embodiment of the present invention, performance of medical procedures such as implantation of medical devices during surgeries. The present invention includes both hardware and software elements that together operate to carry out the various functions embodied within the framework 100. FIG. 1 is an exemplary illustrations of components within the framework 100, and FIG. 2 is an exemplary illustration 200 of a first location 170 and a second location 180 connected via the framework 100.

Hardware elements include one or more devices configured with heads-up displays (HUDs) 110 having semi-transparent displays that are typically worn on one's head, such as augmented reality-enabled glasses 112. Such augmented reality-enabled glasses 112 include one or more sensors and camera(s) and are worn by operating room staff, such as surgeons, scrub technicians, nurses, and anyone else present within an operating room at a first location 170 whom a remote product support specialist may need to communicate. Hardware elements also include one or more display device clients, such as for example mobile devices 120, which may be configured with high-megapixel cameras and high-resolution screens and serve as the host devices for client applications supporting the augmented reality-enabled devices 112. Such mobile devices 120 may, according to one embodiment of the present invention, have a plurality of ports 122 through which the display device clients may connect to augmented reality-enabled glasses 112, Hardware elements may further include additional computing devices 130, which may include tablet computers, mobile telephones, laptop computers, wearable computing devices, and any other computing device that is enabled and configured for communication with other devices within the framework 100.

Hardware elements may further include one or more conferencing servers 160 and associated operating systems that support capture cards for accepting video input from imaging devices 140, such as those used to guide surgeries and other medical procedures. Such imaging devices 140 may include, but are not limited to, flouroscopy machines, arthroscopic/endoscopic cameras and associated feeds, microscopes and associated feeds, surgery-related computer navigation systems, and surgery-related robotic systems. The present invention may also include a dedicated wireless access point(s), configured with security features or password protection, for providing connectivity and communications between the various hardware elements. Hardware elements may still further include devices configured at the second location 180, such as consoles 150 or other display devices that are associated with remote product support specialists.

The framework 100 also includes a plurality of software elements. For example, the present invention includes software running on the augmented reality-enabled glasses 112, the mobile devices 120, on the server(s) 160, and on the additional computing devices 130 such as those being used remotely by the individual providing remote support.

Within the inventory control embodiment of the present invention, the software running on the augmented reality-enabled glasses 112 and on the additional computing devices 130 is configured to enable detection and recognition of physical items within the operating room at the first location 170, such as surgical instruments and surgical implants, for example using object detection and recognition techniques. The software elements include software running in a cloud-based application that is configured to perform functions such as ordering of implants and instruments where the software running on augmented reality-enabled glasses 112 and/or the additional computing devices 130 detects implants are missing from a tray, or the operating room generally, and need to be restocked. The software elements also include software that tracks the movement of items such as the implants, instruments, and/or trays of instruments and implants as they are shipped from external locations, such as warehouses, to hospitals or surgery centers, and further tracks movement of those items from within the hospitals and surgery centers to the operating room for use with patients.

When the present invention is applied to a surgery or medical procedure, the framework 100 is initiated, and a case is scheduled, by first logging into a cloud-based platform and entering details of the surgery or procedure to be performed. This may be done by the surgeon, a surgery scheduler, or a representative in collaboration with the first two; alternatively, it may be done automatically within the cloud-based platform when other triggering information (such as patient, case, or location information) is entered into the system.

Once a case is scheduled, the framework 100 generates a unique identifier, such as a QR code or other type of barcode that comprises a machine-readable optical label that contains information about the item to which it is attached, for the scheduled surgery or procedure. Where the framework 100 includes an inventory control element for all instruments and any implants (where the surgery or procedure involves implanting a medical device) to be used are also associated with the unique identifier. One or more of the surgeon, scheduler, or remote product representative may receive notifications on an application associated with the framework 100 about shipment, arrival and sterilization of instruments, implants, and trays of such instruments or implants upon arrival at the hospital or location where the surgery or procedure is to be performed. On the day of the surgery or procedure, an off-site product representative is able to communicate with the team performing the surgery or procedure remotely and verify the availability and readiness of instruments and implants (such as for example, sterilization) via the application.

To start the remote support system within the framework 100 of the present invention, the additional computing devices 130 (such as tablets), the server 160, and the augmented reality-enabled glasses 112 are powered on, and then all the hardware elements are automatically logged in to the wireless network that is being used (whether it is via a dedicated network or hospital/surgical center network). The unique identifier is provided to the mobile devices 120 and/or additional computing devices 130, or made available to the camera(s) on any of such devices 120 or 130; to connect to the session comprising the planned surgery or procedure, the augmented reality-enabled glasses 112 are manipulated by the wearer to scan the QR code or other unique identifier, either from on the mobile device 120 running the augmented reality client application, one or more of the additional mobile devices 130, or from a texted, emailed or printed version thereof. The remote product representative then receives a notification that augmented reality-enabled glasses 112 are configured for the session and can initiate the session; this initiation of the session may also happen either prior to everyone logging in, or the session may be timed to start at a predetermined time, for example when the surgery or procedure is scheduled.

Once all hardware elements are all connected to the session, the remote product representative is able to control the mobile devices 120, and additional computing devices 130, including audio and camera or video functions such as zooming in or out, and may also be able control similar functions on the augmented reality-enabled glasses 112. For example, the product representative may turn off the screen on the augmented reality-enabled glasses 112, share content on the screen of the augmented reality-enabled glasses 112 to be viewed by the wearer, and communicate via voice or text to the augmented reality-enabled glasses 112. The wearer of the augmented reality-enabled glasses 112 is able to hear the remote product representative, for example via bone conduction audio, audio from speakers on the augmented reality-enabled glasses 112, or connected earpieces (Bluetooth or wired), and also verbally communicate with remote product representatives through microphones; communication may also occur, as noted herein, via gestures made by the wearer, as interpreted by the situational awareness and gesture control function of the framework 100. The remote product representative also is able to communicate with other individuals who can be invited into the session to provide additional support when needed; such individuals can join the session via a link sent to a computer or mobile device to allow the individuals to join.

The framework 100 allows the remote product representative to capture screenshots from any camera associated with the remote support system, such as that configured with the augmented reality-enabled glasses 112, mobile devices 120, or additional computing devices 130. The framework 100 also allows the remote product representative to annotate such screenshots and share those annotations with the augmented reality-enabled glasses 112, mobile devices 120, or additional computing devices 130, as well as annotate live feeds captured, for example, by the augmented reality-enabled glasses 112. The remote product representative may actuate the zoom function on cameras of any of these hardware elements, and capture images or video during the session. All information transmitted between the operating room and the remote product representative is encrypted within the framework 100 to prevent an unauthorized access, at least for protecting patient information and maintaining confidentiality.

The wearer of augmented reality-enabled glasses 112 within the framework 100 of the present invention may utilize a combination of gestures and voice control to control functions thereof. For example, a gesture such as moving the head in a certain manner may open a menu, and items in the menu can be selected by hovering a floating cursor over the menu items, again using specific gestures. Additionally, the head gesture may open a menu and activate a microphone listening for key words to prompt actions in the software (such as selecting menu items, decreasing volume, zooming cameras, switching video feeds, etc.). The framework 100 enables the wearer of the augmented reality-enabled glasses 112 to use gesture controls and voice commands to take pictures or video during the surgery or procedure.

Cameras associated with the augmented reality-enabled glasses 112 and/or the mobile devices 120 and additional computing devices 130 may scan serial numbers and identifying numbers on instruments and implants and record the data as it is needed for billing as well as collecting and recording any information required by regulatory authorities such as the Food & Drug Administration in the United States. At the conclusion of a surgery or procedure, software elements within the framework are able to further record data that is collected in the operating room such as sponge counts, needle counts, sharps counts, instrument counts, implant counts, medications given during a case, blood products given during a case, fluids given during the surgery or procedure, and other commonly recorded data from such surgeries or procedures.

Also, at the conclusion of the surgery or procedure, a surgeon (or other responsible person) has the ability to dictate notes that may be transferred to a broader data management platform in use at the facility where the session occurs. The surgical team may also use one or more of the augmented reality-enabled glasses 112, mobile devices 120, and additional computing devices 130 to scan the instrument trays, implant trays, and tables in the operating room containing instruments and other such items to ensure that item counts are correct and verify which implants need to be re-stocked and re-ordered.

As noted above, the framework 100 of the present invention is not dependent on existing networks in a hospital or ambulatory surgery center where a medical or surgical procedure is to be performed, or on the type of Internet connectivity available at such a facility. The framework 100 may be operable over connectivity provided by either Wi-Fi networks or public data transmission networks, such as the current, widely available 4G network or the 5G network soon to be widely available, and therefore the present invention is not to be limited to any specific type of connectivity for data transmission. Improvements in public data transmission networks such as or the fifth-generation technology standard for cellular networks, enable a transport mechanism for connectivity in the present invention due at least to substantial speed increases from greater bandwidth over prior generations of such networks. Such public data transmission network technology also increases reliability of the framework 100 by utilizing the improved transport mechanism provided by such 5G technology, through increased power capabilities that enable signals generated within the framework 100 to penetrate physical infrastructure and emanate from medical or surgical operating facilities in a stronger manner.

Further, the present invention is designed as a ‘closed’ system, so that the framework 100 is not vulnerable to security breaches, commonly known as hacks, that have the ability to compromise performance, allow malfeasance or interference by outside parties, or theft of patient or facility data. In one example of such a closed system, the present invention utilizes peering technology as a transport mechanism so that no external Internet network is needed to accomplish connectivity between devices. Peering is a process by which separate networks can voluntarily connect and exchange traffic. Peering allows such to directly hand off traffic between each other's customers or clients, without having to rely on a third party to carry that traffic across the Internet for them. In such a peering transport mechanism, a HUD client may be used as hotspot for network connectivity with other devices, such that only devices connected within such a peer-to-peer network are able to communicate within the framework 100.

### Medical and Surgical Procedures

As noted above, the present invention is applicable in particular to medical and surgical procedures that involve implanting medical devices in patients, and where guidance for implanting such devices may be beneficial to the operating team, the patient, or both. Such guidance is provided by product support specialists who offer technical expertise as to the medical device itself and can assist the operating team while the medical device is being implanted in real-time using the framework 100 of the present invention. It is to be understood however that the present is applicable to many other situations, including but not limited to training of medical personnel on the implantation of medical devices. For example, a remote product support specialist in one location may utilize the present invention to instruct surgeons, physicians, nurses, and other personnel in another location on how to use and implant medical devices into bodies of patients. Accordingly, neither the present specification nor the claims are to be limited to any particular use case specifically referenced herein.

### Hardware Components and Software Elements

Different embodiments of the present invention may include different hardware components and software elements. For example, in one exemplary embodiment of the present invention, specific hardware components may include one or more servers or processors, either locally situated or available in a cloud computing environment as noted above.

Other specific hardware components may include, also as noted above, augmented reality-configured display devices, such as an optical head-mounted display (OHMD) or other head-up displays (HUDs), such as for example Epson Moverio BT-35E, Vuzix M4000, Jorjin J7EF, Lenovo A6, etc. It is to be understood however that any OHMD or HUD, or combination thereof, in any number, may be used, and the present disclosure is not intended to be limited to any one type of augmented reality-enabled display device. The hardware components may further include an Android-based, iOS-based (or any other operating system) mobile device for a HUD client connected to a HUD device, and other mobile or desktop computing devices, such as for example other mobile devices (such as iPhones), iPads, other tablets, and other personal computers or Android or other PC Windows/Mac (web) clients, and regardless of the operating system on which those other devices perform. Wi-Fi access points and cellular network hotspots are still other hardware components that may be part of the framework of the present invention.

The augmented reality-configured display devices generate input data for processing in the software elements of the framework 100, at least in the form of signals responsive to sensed gestures and/or movement of wearers of those devices, via the gesture interpretation and control system described below. This input data is processed and analyzed in one or more algorithms as described below, which perform multiple data processing and mathematical functions within the framework 100.

The framework 100 of the present invention may, as noted above, also include specific software elements within an overall software framework operating in conjunction with these hardware components. The framework 100 is embodied in one or more systems and methods in which the software elements are executed within, or at the direction of, a plurality of data processing modules that are components within a computing environment that includes one or more processors which govern and control the hardware components and software elements described herein. The one or more processors are configured to execute program instructions or routines that are stored on or accessed from a storage medium, non-transitory or otherwise, to perform the modules, components, and data processing and mathematical functions described herein, and embodied within the one or more data processing modules configured to carry out such data processing and mathematical functions.

Specific software elements may include server software code and a HUD client application (such as Android), and may also include elements that integrate software development kit(s) for specific augmented-reality display devices for accessing display functions, and gesture interpretation and control system software for enabling accelerometer/gyroscope subsystems that are sensors for gesture control. Software elements may also include one or more specific client applications, for example for a product support specialist control panel and other tools that facilitate participation of product support specialists in collaboration sessions. Other software elements include one or more algorithms configured to execute the data processing and mathematical functions that at least interpret gestures and movement within the gesture interpretation and control system.

### Description of Operation

Regardless of the network configuration, and regardless of the specific hardware components or the nature of software elements described above and/or utilized therein, the architecture of the present invention is designed for straightforward, easy-to-use performance as a medical or surgical augmented-reality assistant for support in medical and surgical situations, in a plug-and-play approach. The framework 100 operates in principle as follows. A scrub tech, nurse, assistant, physician, or any other in-setting individual dons a HUD device (or any augmented reality-enabled display device), connects to a mobile device acting as a HUD client, and launches the HUD client application. Upon launch, the HUD client and HUD client application perform network tests to determine whether to connect securely to a local server or cloud-based server, based on the assigned IP address. Pending network confirmation, a secondary test is executed to detect and initialize the wearable device's software development kit (SDK), and perform diagnostic testing on accelerometers, gyroscopes, and other sensors configured with the augmented reality-enabled display device. Any failure in these detection tests is noted to the user and corrected and stored if the present invention includes and is coupled to a database or other data storage component. Once preliminary checks are successful, the HUD client application self-registers with the conferencing server 160 and is ready to accept inbound calls from the product support specialist client and other. The HUD client is then placed in a ready/waiting state by default until all systems are initialized for connection status.

With the HUD client launched and registered with the conferencing server 160, the product support specialist client application is launched and auto connected to the secure conferencing server 160 and provided a list of clients to connect with. Connection requests are then made to the HUD client and initiated. These connections remain in a connected state until the connection is terminated by either party. Additional clients may be invited to join the ongoing conference, either in a watch/listen only mode, or by invitation for full participation in a multi-person/device conference.

The framework 100 includes, as noted above, both hardware components and software elements configured to interpret movement of the individual(s) wearing HUD devices. Maintaining a sterile environment in an operating facility is a health and safety issue for all involved, and present invention promotes this objective by enabling the HUD client to infer actions for motion using a touch-free, fully heads-up gesture interpretation and control system. The accelerometer, gyroscope, and other sensors of the augmented reality-enabled display device are polled and continuously measured and analyzed. This is done at least by initializing a resting/steady point for “North”, and periodically calibrating this resting/steady point, to detect and interpret actions such as ‘swipe right’, ‘swipe left’, and tilted up/elevated ‘invoke menu’ indications. For users unfamiliar with operation of the HUD device, upon initial launch of an application within the present invention, a brief tutorial display appears to demonstrate to the user how to make these actions and allows them to practice the various maneuvers. This also ensures the HUD device is fully initialized and ready to be used.

Actions interpreted by the gesture interpretation and control system may include ‘head swipe left’, which action enables/disables the HUD display in the glasses (screen on/off), and otherwise to ignore in-bound calls (visual notification is provided to the wearer). A ‘head swipe right’ action answers in-bound calls (also with visual notification). A ‘head tilted up/elevated’ action displays menus, and moves a cursor to invoke additional actions such as screen capture, camera controls, mute/unmute, etc. This action also invokes a menu once a certain number of seconds of position (configurable—default 5) being held above the y axis have passed, without floating to the left or to the right. It is to be noted that many other actions by the wearer or the user of the HUD device may induce responses by the gesture interpretation and control system, and the framework 100 may be configured to respond accordingly.

The HUD device may include, as noted above, one or more sensors to detect gestures of a wearer of the HUD device, as well as software elements specifically configured to detect and identify movement of the wearer of the HUD device. These may include, as noted herein, an accelerometer, a gyroscope, and any other sensor capable of detecting movement of an augmented reality-configured display device.

The product support specialist client is also configured to perform many functions within the present invention. For example, product support specialist client may initiate and terminate conference calling with the HUD client. The product support specialist client may also be configured to control operation of a camera by turning it on or off and/or enabling or disabling certain functions thereof and may also perform mute/unmute functions for both itself and the HUD client, take snapshot images of a media stream, and other functions. This is accomplished using a dedicated client for the PSS, or a web client that can invoke actions directly to the HUD client via the server 160. For example, the local HUD client may control the local camera, but the product support specialist client may enable or disable the HUD camera from streaming.

The software elements within the framework 100 may comprise of different modules, components, and features. As described above, the server(s) 160 may be deployed in different configurations. The server(s) 160 may be fully contained, locally on-site, and using a portable computer or laptop with its own access point for establishing private P2P communication with the client(s). The software elements for enabling server functions may comprise different approaches, depending at least on the version and timing of the solution.

One exemplary software implementation within the framework 100 leverages existing platforms and application programming interfaces (APIs), for example Twilio, Zoom, or PubNub. Alternatively, a proprietary technology stack may be utilized to support the framework 100 and enable specific features described herein. It is therefore to be understood that both existing and proprietary software components may be utilized to accomplish the various aspects of the present invention.

Regardless of the approach (off-the-shelf or proprietary), the software elements of the framework 100 leverage existing standards and protocols (such as WebRTC and other VoIP signaling standards, security protocols such as sRTP and sRTMP encryption, and other existing best practices) to ensure enterprise-grade solutions for the establishment of reliable communication between clients through the server 160. The present invention is designed to operate within a software framework, whether deployed in a cloud-based environment or locally, that is designed to have a footprint that is minimized to provide essential P2P communications. This is accomplished at least in part by utilizing server code written in a compact manner, and that leverages MySQL databases and other frameworks, to support local, on-premises portable server devices.

Aspects of the software framework supporting the HUD client is written for Android operating systems and integrates APIs for software development kits for existing devices such as Epson's Moverio BT-35E, Lenovo's ThinkReality A6, and other OEM smart glasses. The software framework may utilize Twilio for backend processes and integrate Twilio's video software development kit. Alternatively, Twilio may be substituted with direct, proprietary software for the conferencing server 160 and communication with the product support specialist client.

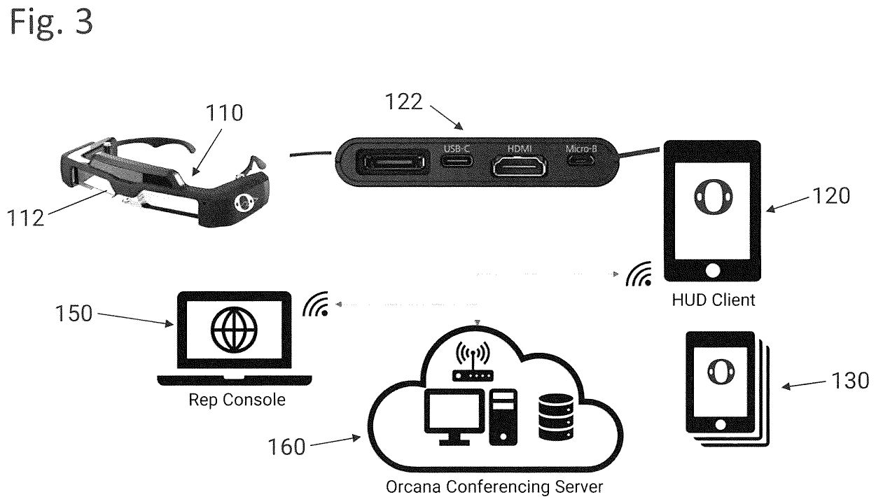

FIG. 3 illustrates various aspects of the present invention. FIG. 3 shows exemplary hardware components of the framework 100 disclosed herein. FIG. 3 illustrates an exemplary local configuration of how the hardware elements of the framework 100 may be implemented.

As noted above, the framework 100 of the present invention may also be styled as a system and method of remotely guiding an implantation of device during a medical or surgical procedure, such as for example an artificial heart, or an artificial joint such as a knee or hip. It is to be understood that any type of medical device to be implanted into a patient's body may be remotely guided by product support specialists who are not physically present the operating facility using the present invention. The present invention is therefore applicable in any operating environment where product support specialists may assist with the implantation of such devices using the various hardware components and software elements described herein.

### Image Processing and Object Detection for Accounting of Materials

In one embodiment of the present invention, the framework 100 also includes the ability to detect and identify objects before, during, and after a medical or surgical procedure for the purpose of tracking materials, instruments and devices used, monitoring their presence and use, and verifying one or more characteristics relative to their use in such a procedure. The present invention may include image processing and object detection capabilities for such tracking, monitoring, and verifying, such as for example pixel analysis techniques and other approaches to characterizing pixels in images that enables an automated detection and identification of instruments in an operating facility, regardless of whether such automated detection and identification occurs, before, during, or after a procedure. Such tracking, monitoring, and verifying may be useful for inventory control, and ensuring that all necessary components are present and ready prior to an invasive procedure. It is often the situation that instruments needed for certain procedures are missing or unavailable before a procedure, and also often the situation that the medical device itself to be implanted is either not available or missing certain parts. Ascertaining such situations and remedying them prior to a procedure helps to avoid wasting resources such as availability of facilities and professionals' time, as well as minimizing the impact on patients themselves.

In such a tracking, monitoring, and verifying embodiment, the present invention may also provide alerts or notifications relative to the materials, instruments, or devices used. For example, alerts and notifications may be provided that such materials, instruments, or devices are present and ready for use. Many other examples are also possible, such as that one or more materials, instruments, or devices are not present prior to a procedure, present but in need of sterilization prior to a procedure, missing and need to be provided prior to a procedure, or missing and unaccounted for following a procedure. It is to be understood that any type of alert or notification attendant to tracking, monitoring, or verifying materials, instruments, or devices, whether aural, visual, or textual, are possible and within the scope of the present invention.

### Additional Features

In addition to the hardware components and software elements shown therein, and the specific features described above, many other features may be included within the present invention. For example, the present invention may include an “auto” registration feature, which enables clients to automatically register with the ‘nearest’ server, as defined by one or more algorithms. This ensures that all clients connected to the conferencing server 160 are able to see each other and communicate. The present invention may also be scalable to many clients, and the ‘auto’ registration may enable multiple clients to register at the same time and/or for the same session or conference, so that, for example, groups and other viewers/participants may be able to conference together, rather than just an HUD client and a PSS client.

The framework 100 of the present invention may also include a profile management feature, to ensure that only trusted participants are able to join a collaboration session. This feature is useful to identify, register, confirm, and authorize PSS client participation, particularly where outbound emails are sent, or where outbound data transmission is required, from the server 160 or through the server 160. The present invention may further include a listing feature that displays a server-side contact list of devices available, so that all connected clients are shown for identification and security purposes.

Video and voice calling is a further feature that may be included within the present invention. In such a feature, the PSS client is able to initiate call requests with the HUD client. The HUD client can accept or reject inbound call requests. Additionally, the HUD client may also be able to initiate voice and video calls. P2P or group calls, both voice and video, are another feature possible within the present invention between PSS clients and the HUD client.

The framework 100 may also include a text-based chat feature. For example, the PSS client can send text messages to the HUD client; the HUD Client may be configured to auto-display such messages, as well as display or clear incoming messages using particular gestures.

The framework 100 may also include a screen sharing feature. In this feature, the PSS client may share screens with the HUD client, for example where product support specialists need to display materials or demonstrate usage of a particular product. The screen sharing feature may further include interactive capability, so that the product support specialist can manipulate his or her screen using touch functionality, and the manipulations will appear on the display of the HUD device.

Screen sharing in the framework 100 includes communicating and transmitting data representing a display from the remote, or second, location where the product support specialists are located (and also, any other remote location where additional participants in a collaboration session or located), to the first location where the surgery or procedure is taking place, and vice versa so that users remote from each other can see, for example, manipulations being performed on an image or video. This means that in addition to live streams, images, and messages, data communicated within the framework 100 may also include the display seen at one location so that same or similar screen is seen at the other location, as well as manipulations being performed on the screen being shared. This screen-sharing feature may include screen shots, screen shares, annotations, video-sharing, animations, and other data that one user wishes to share with another user.

The present invention may also provide the ability to communicate animations from product support specialists to medical personnel wearing a HUD device. For example, a product support specialist may send an animation (or portion of an animation) showing specific steps in an implantation of a medical device as part of remote guidance for such an implantation. Photos or stills, animated or otherwise, may also communicated across the framework 100 of the present invention. For example, a product support specialist may be able to manipulate a photo or still, such as by drawing a circle around a picture of a medical device, to indicate a specific component of the implantable device that the medical personnel must attend to during such remote guidance. It is to be understood that transmission of images, animated or otherwise, are two-way within the present invention. For example, images captured by the HUD device may be viewable by remotely situated product support specialists on their console or graphical user interface, and this may be used to further enable such remote guidance of the implantation of the medical device.

As noted above, the present invention may include several security features, such as for example encryption of person-to-person communications. All connections in the framework 100 are encrypted end-to-end, using any known encryption protocol or standard. For example, connections may be encrypted using AES-256-bit encryption, or SHA-256-bit encryption.

Yet another feature of the framework 100 may be noise cancellation. This may assist users during a session by preventing noise when the wearer of a HUD device speaks and may also assist wearers by cancelling out ambient noise in the operating facility to focus on the voice of the product support specialist or any other remote speaker. Still further features that are possible within the framework 100 include video and audio participant detection and recognition, video, audio, and textual alerting, session recording and cloud storage of recorded material, external communications such as email and via text messaging and chatting via social media channels, and linking to participant accounts for email, text, and social media communications. The present invention may further include historical logging of session activity, and scheduling of updates to check for client connectivity and performance, software updates, and any other system maintenance.

The present invention may also include or access a machine learning modeling layer that is configured to perform various functions within the present invention. These may include interpreting gestures or movement of wearers of augmented reality-configured display devices and diagnosing or solving problems that arise during performance of a medical or surgical procedure. The machine learning modeling layer may be comprised of one or more neural networks that further evaluate input data, such as information generated by augmented reality-configured display devices, to perform specific algorithms (and increase their accuracy) performed by the conferencing server 160 attendant to these functions. The present invention therefore contemplates that one or more techniques of artificial intelligence may be applied to the data processing and mathematical functions described herein and may themselves constitute data processing and mathematical functions performed within the framework 100.

The systems and methods embodying the present invention may be implemented in many different computing environments. For example, the framework 100 may be implemented in conjunction with one or more special purpose computers, a programmed microprocessor or microcontroller and peripheral integrated circuit element(s), an ASIC or other integrated circuit, a digital signal processor, electronic or logic circuitry such as discrete element circuit, a programmable logic device or gate array such as a PLD, PLA, FPGA, PAL, and any comparable means. In general, any means of implementing the hardware components and software elements illustrated herein can be used to implement the various aspects of the present invention. Exemplary hardware that can be used for the present invention includes computers, handheld devices, telephones (e.g., cellular, Internet enabled, digital, analog, hybrids, and others), and other such hardware. Some of these devices include processors (e.g., a single or multiple microprocessors), memory, nonvolatile storage, input devices, and output devices. Furthermore, alternative software implementations including, but not limited to, distributed processing, parallel processing, or virtual machine processing can also be configured to perform the methods described herein.

The systems and methods of the present invention may also be partially implemented in software that can be stored on a storage medium, executed on programmed general-purpose computer with the cooperation of a controller and memory, a special purpose computer, a microprocessor, or the like. In these instances, the systems and methods of this invention can be implemented as a program embedded on personal computer such as an applet, JAVA® or CGI script, as a resource residing on a server or computer workstation, as a routine embedded in a dedicated measurement system, system component, or the like. The system can also be implemented by physically incorporating the system and/or method into a software and/or hardware system.

Additionally, the data processing functions disclosed herein may be performed by one or more program instructions stored in or executed by such memory, and further may be performed by one or more modules configured to carry out those program instructions. Modules are intended to refer to any known or later developed hardware, software, firmware, artificial intelligence, fuzzy logic, expert system or combination of hardware and software that can perform the data processing functionality described herein.

In closing, it is to be understood that, although aspects of the present specification are highlighted by referring to specific embodiments, one skilled in the art will readily appreciate that these described embodiments are only illustrative of the principles of the subject matter disclosed herein. The specific embodiments are not intended to be exhaustive or to limit the invention to the precise forms disclosed. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions, and sub-combinations thereof can be made in accordance with the teachings herein without departing from the spirit of the present specification. It is therefore intended that the scope of the invention is not to be limited by this detailed description. Furthermore, it is intended that the following appended claims and claims hereafter introduced are interpreted to include all such changes, modifications, permutations, alterations, additions, subtractions, and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified, thus fulfilling the written description of all Markush groups used in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. For instance, as mass spectrometry instruments can vary slightly in determining the mass of a given analyte, the term “about” in the context of the mass of an ion or the mass/charge ratio of an ion refers to +/−0.50 atomic mass unit. At the very least, and not as an attempt to limit the application of the doctrine of equivalents to the scope of the claims, each numerical indication should at least be construed considering the number of reported significant digits and by applying ordinary rounding techniques.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as, e.g., “first,” “second,” “third,” etc. —for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising”, variations thereof such as, e.g., “comprise” and “comprises”, and equivalent open-ended transitional phrases thereof like “including,” “containing” and “having”, encompass all the expressly recited elements, limitations, steps, integers, and/or features alone or in combination with unrecited subject matter; the named elements, limitations, steps, integers, and/or features are essential, but other unnamed elements, limitations, steps, integers, and/or features may be added and still form a construct within the scope of the claim. Specific embodiments disclosed herein may be further limited in the claims using the closed-ended transitional phrases “consisting of” or “consisting essentially of” (or variations thereof such as, e.g., “consist of”, “consists of”, “consist essentially of”, and “consists essentially of”) in lieu of or as an amendment for “comprising.” When used in the claims, whether as filed or added per amendment, the closed-ended transitional phrase “consisting of” excludes any element, limitation, step, integer, or feature not expressly recited in the claims. The closed-ended transitional phrase “consisting essentially of” limits the scope of a claim to the expressly recited elements, limitations, steps, integers, and/or features and any other elements, limitations, steps, integers, and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps, integers, and/or features specifically recited in the claim, whereas the meaning of the closed-ended transitional phrase “consisting essentially of” is being defined as only including those elements, limitations, steps, integers, and/or features specifically recited in the claim and those elements, limitations, steps, integers, and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such, the embodiments described herein or so claimed with the phrase “comprising” expressly and unambiguously provide description, enablement, and support for the phrases “consisting essentially of” and “consisting of.”

All patents, patent publications, and other references cited and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard is or should be construed as an admission that the inventors are not entitled to antedate such disclosure by virtue of prior invention or for any other reason. All statements as to the date or representation as to the contents of these documents are based on the information available to the applicant and do not constitute any admission as to the correctness of the dates or contents of these documents.

Lastly, the terminology used herein is for the purpose of describing particular embodiments only and is not intended to limit the scope of the present invention, which is defined solely by the claims. Accordingly, the present invention is not limited to that precisely as shown and described.

1. A method for remotely guiding implantation of a medical device in a medical or surgical procedure, comprising:
launching a display device client application to initiate a connection to a conferencing server for a collaboration session involving implantation of a medical device into a patient's body at a first location, and to detect and initialize an augmented reality-enabled display device and a display device client;
connecting one or more additional computing devices at the first location to the conferencing server;
launching a product support specialist client application to connect a product support specialist client to the conferencing server;
initiating a connection request to the display device client to enable the product support specialist client to join the collaboration session from a second location that is remote from the first location;
initiating a live stream of content captured by the augmented reality-enabled display device and content captured by one or more additional computing devices at the first location, and communicating the live stream from the first location and the second location;
communicating annotations of the live stream of the content performed by the remote product support specialist from the second location to the first location;
sensing and interpreting gestures made with the augmented reality-enabled device and by a wearer to determine a control of actions within the collaboration session from the gestures, the control of actions including manipulating functions of the augmented-reality-enable device and communicating with the remote product support specialist based on the gestures, and transmitting data relative to the control of the actions made by the wearer from the gestures to the product support specialist client; and
transmitting voice and textual interactions between the augmented reality-enabled display device and the product support specialist client.

2. The method of claim 1, wherein the live stream includes live video, recorded video, and images.

3. The method of claim 1, wherein the launching a display device client application further comprises generating a unique collaboration session identifier and transmitting the unique collaboration session identifier to one or more of the display device client and at least one of the additional computing devices.

4. The method of claim 3, wherein a wearer of the augmented reality-enabled device initiates the collaboration session by manipulating the augmented reality-enabled device so that camera configured with the augmented reality-enabled device captures the unique collaboration session identifier.

5. The method of claim 1, further comprising applying one or more image processing techniques to detect and identify one or more objects representing materials, instruments, or devices attendant to the implantation of the medical device at the first location.

6. The method of claim 1, wherein the display device client is a mobile computing device, and the additional computing devices are tablet computers configured with cameras to capture one or more of videos and images within the first location.

7. The method of claim 1, further comprising communicating animations from the second location to the first location, and displaying the animations on the augmented reality-enabled display device.

8. A system for remote medical and surgical support, comprising:
a display device client operatively coupled to an augmented reality-enabled display device that is configured to capture a live stream of content relative to implantation of a medical device into a patient's body, the display device client configured to sense and interpret one or more gestures of the wearer of the augmented reality-enabled display device;
a conferencing server configured to initiate and manage a collaboration session involving the implantation of the medical device at a first location, at least between a wearer of the augmented reality-enabled display device and a product support specialist during performance of the implantation in an operating theater at the first location;
one or more additional computing devices operatively coupled to the conferencing server and configured to capture additional content relative to the implantation of the medical device at the first location; and
a product support specialist client associated with the product support specialist at a second location remote from the first location, the product support specialist client configured to display the live stream and the additional content transmitted from the display device client during the collaboration session,
wherein the one or more gestures are interpreted to determine a control of actions within the collaboration session, the control of actions including manipulating functions of the augmented-reality-enable device and communicating with the remote product support specialist based on the gestures, and wherein messages representing the control of the actions made by the wearer and with the augmented reality-enabled device from the one or more gestures are transmitted from the first location to the second location, annotations of the live stream performed at the product support specialist client are transmitted from the second location to the first location, and verbal and textual messages are transmitted between the second location and the first location.

9. The system of claim 8, wherein the live stream captured by the augmented reality-enabled display device includes live video, recorded video, and images.

10. The system of claim 8, wherein the display device client application generates a unique collaboration session identifier, and transmits the unique collaboration session identifier to one or more of the display device client and at least one of the additional computing devices.

11. The system of claim 10, wherein a wearer of the augmented reality-enabled device initiates the collaboration session by manipulating the augmented reality-enabled device so that camera configured with the augmented reality-enabled device captures the unique collaboration session identifier.

12. The system of claim 8, wherein one or more image processing techniques are applied to detect and identify one or more objects representing materials, instruments, or devices attendant to the implantation of the medical device at the first location.

13. The system of claim 8, wherein the display device client is a mobile computing device, and the additional computing devices are tablet computers configured with cameras to capture one or more of videos and images within the first location.

14. The system of claim 8, wherein animations are communicated from the second location to the first location, and displayed on the augmented reality-enabled display device.

15. A method for performing remote medical and surgical support in an operating environment, comprising:
initiating a collaboration session involving implantation of a medical device into a patient's body at a first location, by connecting a display device client application to a conferencing server, and detecting and initializing an augmented reality-enabled display device associated with the display device client;
launching a product support specialist client application to connect a product support specialist client at a second location remote to the first location to the conferencing server;
initiating a connection request to the display device client to enable the product support specialist client to remotely join the collaboration session;
identifying and joining additional clients to the collaboration session that represent one or more additional computing devices at the first location;
sensing one or more gestures of a wearer of the augmented reality-enabled display device;
determine a control of actions within the collaboration session from the one or more gestures made by the wearer and with the augmented reality-enabled device, the control of actions including manipulating functions of the augmented-reality-enable device and communicating with the remote product support specialist based on the one or more gestures;
generating messages to the product support specialist client representing the one or more gestures of the wearer of the augmented reality-enabled display device; and
communicating information between the first location and the second location relative to the collaboration session, the information including
data representing the control of the actions made by the wearer of the augmented reality-enabled device,
data from the first location to the second location that least include a live stream of content captured by the augmented reality-enabled display device and one or more of the additional computing devices,
data from the second location to the first location that at least include annotations of the live stream performed at the product support specialist client, and
data that includes verbal and textual messages between the product support specialist client and the display device client.

16. The method of claim 15, wherein the live stream of content includes live video, recorded video, and images.

17. The method of claim 15, further comprising generating a unique collaboration session identifier and transmitting the unique collaboration session identifier to one or more of the display device client and at least one of the additional computing devices.

18. The method of claim 17, wherein the wearer of the augmented reality-enabled device initiates the collaboration session by manipulating the augmented reality-enabled device so that camera configured with the augmented reality-enabled device captures the unique collaboration session identifier.

19. The method of claim 15, further comprising applying one or more image processing techniques to detect and identify one or more objects representing materials, instruments, or devices attendant to the implantation of the medical device at the first location.

20. The method of claim 15, wherein the display device client is a mobile computing device, and the additional computing devices are tablet computers configured with cameras to capture one or more of videos and images within the first location.

21. The method of claim 15, wherein the communicating information between the first location and the second location relative to the collaboration session further comprises communicating animations from the second location to the first location,

22. The method of claim 21, further comprising displaying the animations on the augmented reality-enabled display device.
